SEQUENCE LISTING (5 sequences)

SEQ ID NO: 1 - DNA, 670 bp, Enterobacter sp. SGT 06-1
gataatcgcatacgtcgcagaccaagagggggaccttcgggcctcttgccatcagatgtgcccagatgggattagctagtaggcggggtaatggcccacctaggcgacgatccctagctggtctgagaggatgaccagccacactggaactgagacacggtccagactcctacgggaggcagcagtggggaatattgatcaatgggcgcaagcctgatgcagccatgccgcgtgtgtgaacaaggccttcggattgtaaatcgctttctccgggtaggaaggcctgccggttaataaccgtgccgattgactttacccgcagaagaagcaccggctaactccgtgccgcacccgcttcctccccgagggtgcaagcgttaatcggaattaccttcttcaaagcgcacgcatggctgctgtcaagttggatgtgaaatccccgggctcaacctgctaactgcattcgaaactgggaggctggagtctcgtagagggaggtggaattcctcgtgtaccggtgaaatgcctacacctctggaggactatccccggccaacgcggtctcctggactaagactgactctcaggtgcaaaaccgtggggaccccacttgattatataccctggtagtccactccgctaacgatgtcaacttgatgctcccttcaaa

SEQ ID NO: 2 - DNA, 20 bp, Artificial Sequence
  Primer for RecA gene analysis of SGT 06-1
ggtaaagggtctatcatgcg

SEQ ID NO: 3 - DNA, 22 bp, Artificial Sequence
  Primer for RecA gene analysis of SGT 06-1
ccttcaccatacataatttgga

SEQ ID NO: 4 - DNA, 20 bp, Artificial Sequence
  Forward primer SGT-UNI04-fw3
tggagggggataactactgg

SEQ ID NO: 5 - DNA, 18 bp, Artificial Sequence
  Reverse primer SGT-UNI04-rv2
ggcacaacctccaagtcg

(19) United States
(12) Patent Application Publication
(10) Pub. No.: US 2009/0029433 A1
(43) Pub. Date: Jan. 29, 2009

(54) HYDROGEN PRODUCING MICROORGANISM USEFUL FOR ENERGY GENERATION FROM DIVERSE CARBOHYDRATES

(75) Inventors: Elmar Schmid, Escondido, CA (US); James Warner Gibson, Escondido, CA (US)

Correspondence Address: Patentique PLLC, P.O. Box 5803, Bellevue, WA, 98006, US

(73) Assignee: Dr. Elmar Schmid and James Gibson, Escondido, CA (US)

(21) Appl. No.: 11/829,599

(22) Filed: Jul. 27, 2007

(51) Int. Cl. C12P 3/00; C07H 21/00; C12N 1/00; C12N 15/00; C12N 15/01; C12N 15/74

(52) U.S. Cl. 435/168; 435/243; 435/440; 435/443; 435/444; 435/446; 435/448; 435/472; 435/473; 536/24.32

(57) ABSTRACT

The disclosed invention relates to an isolated hydrogen gas producing microorganism, termed Enterobacter sp. SGT 06-1™.

### FIELD OF THE INVENTION

The disclosed invention relates to the field of hydrogen gas production utilizing suitable microorganisms as an environmentally friendly alternative of energy production for human society. The disclosure describes an isolated and genetically unique microorganism, termed Enterobacter sp. SGT 06-1™. The microorganism generates high amounts of hydrogen gas in the presence of different cellulosics- and hemicellulosics-derived sugars as well as alcoholic sugars as carbon source which allows utilization of hydrogen energy, e.g. in form of electricity and heat, at sites with traditionally high generation of cellulosics- and hemicellulosics and other renewable bio-waste materials.

### BACKGROUND OF THE INVENTION

In view of increasing costs for non-renewable and depleting fossil energy resources, such as oil, coal and natural gas, and of the rising discharge of the combustion-derived green house gas carbon dioxide (CO₂) into the atmosphere, there is an increasing interest and pressing need in the replacement of carbonized fuels by cleaner, environmentally friendly alternative fuels. Amongst different discussed and tested renewable energy fuels, including ethanol and bio-diesel, hydrogen gas (H₂) is considered to be a prime alternative fuel candidate. The renewed global interest in H₂ as an alternative future fuel is spurred by the fact that combustion of hydrogen gas results in the formation of water with no emission of the green house gas carbon dioxide (CO₂) as opposed to burning fossil fuels, biodiesel and ethanol. Moreover, hydrogen gas can be directly converted to electricity with high conversion efficiencies using fuel cell technologies. Since industrial scale hydrogen gas production from fossil fuels, i.e. by steam reforming or coal gasification, is accompanied with carbon dioxide, carbon monoxide and nitrogen oxides (NOₓ) generation, there is a high interest for alternative hydrogen gas-generating technologies. Even though several hydrogen energy concepts and technologies have been innovated, developed and introduced into the market, it is clearly understood, that there will only be a real benefit for global carbon dioxide abatement when, 1) either the fossil fuel-derived CO₂ is sequestered during the production process or 2) when hydrogen gas is produced from renewable resources which assures a closed global carbon cycle. Various renewable resources, including municipal solid wastes (MSW), food and packaging wastes, agriculture and forestry wastes, have been studied in the past decades as a cheap, abundant and carbon-rich feedstock for hydrogen production with the help of microorganisms (Hallenbeck, P. C. Water Sci Technol. 52(1-2):21-29 (2005); Nandi, R. et al., Critical Reviews in Microbiology 24(1):61-84 (1989); Roychowdhury, S. et al., Int. J. Hydrogen Energy 13:407ff (1988)). None of the described microorganisms and methods lead to a successful functioning industrial scale bio-hydrogen production system. The microorganisms which have been studied for industrial scale production of hydrogen gas can be roughly classified into two major groups: 1) photosynthetic organisms, such as the photosynthetic Rhodobacter sp. bacteria, the cyanobacterium Oscillatoria sp. and the green algae Chlamydomonas reinhardtii, and 2) heterotrophic bacteria.

The use of microorganisms for large scale production of hydrogen gas as an environmentally clean fuel has many advantages over the current industrial scale generation of hydrogen gas from fossil fuels, e.g. gasification of coal. Microbial hydrogen production can be fueled with renewable biomass and/or derivatives thereof and can be conducted at ambient temperatures and pressures under comparatively low cost conditions (Hallenbeck, P. C. Water Sci Technol. 52(1-2):21-29 (2005); Nandi, R. et al., Critical Reviews in Microbiology 24(1):61-84 (1989)). Moreover, microbial hydrogen production is—with the exception of some thermophilic bacteria—not accompanied with the release of toxic and/or noxious gases, such as carbon monoxide (CO) and hydrogen sulfide (H₂S).

To date, however, no microorganism with a suitably high and sustainable hydrogen gas productivity has been reported that would allow industrial scale generation of biohydrogen energy. No process or technique has been described which allows utilization of a suitable hydrogen gas generating candidate microorganism for long term and low cost production of hydrogen gas. Hydrogen gas generation with the help of photosynthetic microorganisms requires rather expensive incubation vessels with large, light-exposed surface areas and large quantities of increasingly expensive water. Effective hydrogen production in photosynthetic microorganisms is further hampered by low hydrogen production rates due to concomitantly released oxygen gas. Heterotrophic bacteria have the advantage that they do not need solar energy and elaborate fermentation vessels for hydrogen production, but they are dependent on a suitably cheap feedstock to assure low cost hydrogen production. Furthermore, the feedstock has to be supplied continuously and under contamination-free conditions to assure long term generation of hydrogen gas in the comparatively low cost fermentation vessels. Despite the fact that high and continuous hydrogen production has been shown for a series of heterotrophic microorganisms, including Klebsiella oxytoca (Beneman, J. Nature Biotechnol. 14:1101ff (1996)), Thermotoga neapolitana (Van Niel, E. W. J. et al., Int. J Hydrogen Energy 27:1391-1398 (2002)) and Clostridium beijerinckii (Taguchi, F. et al., U.S. Pat. No. #5,350,692 (Sep. 27, 1994)), under experimental lab conditions and with purified glucose as the feedstock, no long term generation of hydrogen gas has been reported for any of the known hydrogen producers with cheap waste feedstock to date. Continuous high hydrogen production by known anaerobic hydrogen producing bacteria, such as Clostridia sp. and Thermotoga sp., is hampered by the introduction of oxygen gas, a growth toxin to these microorganisms, usually carried in with the continuously supplied feedstock. Another major obstacle which prevented the successful industrial scale use of heterotrophic microorganisms for cost-effective generation of hydrogen gas from cheap waste feedstock is the high risk of contamination of the reaction vessel with the continuously supplied feedstock material. Therefore, a facultative anaerobic and robust microorganism with high tolerance for oxygen levels and that would allow continuously high hydrogen production from cheap feedstock would be advantageous for an aspired industrial-scale biohydrogen production system. Furthermore, even though a series of mesophilic and moderate thermophilic microorganisms have been studied intensively for quantitative bio hydrogen production from glucose, sucrose and/or starch-containing feedstock, no reports exist for a more versatile bacterium capable of generating high amounts of hydrogen gas from other cellulosics and hemicellulosics-derived carbohydrates, most namely cellobiose, arabinose, rhamnose, xylose and galactose.

Plant-derived cellulose-containing materials (often referred to as cellulosics) are the single most abundant renewable carbon source on earth and are annually produced by photosynthetic organism, such as grasses, shrubs and trees, on a Giga ton scale. Globally green plants convert about 190 Giga tons of carbon dioxide into biomass annually. Industry-processed cellulosics, such as paper, newsprints, card board, and shopping bags, make up more than 40% of all municipal solid waste, a waste stream that to the vast extent ends up in land fills.

Therefore, a metabolically versatile microorganism capable of generating hydrogen gas from diverse cellulosics- and hemicellulosics-derived carbohydrates and other carbon compounds, could make a significant contribution to help conserve existing landfill space and also to generate clean energy from agricultural biomass waste.

### BRIEF SUMMARY OF THE INVENTION

The disclosure is based in part on an isolated microorganism, referred to as SGT 06-1™ herein. The microorganism produces hydrogen gas (or molecular hydrogen, H₂) and belongs to the bacterial family of enterobacteriaceae, a ubiquitous and versatile group of gram-negative, facultative anaerobic bacteria. Enterobacteria are known to be metabolically versatile and are able to gain cell energy via respiratory (aerobic) or fermentative (anaerobic) degradation of a wide variety of different carbon containing molecules as starting materials. Enterobacteria, which commonly occur in soil, water, sewage, food and are also found as normal intestinal inhabitants of humans and animals, are well studied and known to catabolize D-glucose and other carbohydrates, including L-arabinose, cellobiose, maltose, D-xylose, L-rhanmose, D-mannitol, D-sorbitol and trehalose, with the production of organic acids and gas. Glucose can be derived from many sources, but it is very abundant in green plants and in other renewable biomass-derived materials where it usually appears in the form of the disaccharide sucrose and of the polysaccharides starch and cellulose (FIG. 8). Other monosugars, most prominently arabinose, xylose, galactose and rhamnose are common components of the hemicellulose and pectin fraction of renewable biomass, e.g., green plants and other phototrophic organisms (FIG. 8).

Thus in one aspect, the disclosure includes a hydrogen producing microorganism as described herein. One non-limiting example of a microorganism of the disclosure is a microorganism comprising a 16S rDNA sequence fragment represented by SEQ ID No: 1 (Table 5). The disclosure thus includes a microorganism of the enterobacteriaceae family which generates high amounts of hydrogen gas from carbohydrates derived from a diverse range of starch, cellulose, and hemicellulose containing materials. In some embodiments of the disclosure, the microorganism utilizes one or more of the carbohydrates shown in FIG. 8 to generate hydrogen gas. In other embodiments, a combination of two or more of the carbohydrates is utilized by the microorganism.

In a second aspect, the disclosure includes a method of culturing a microorganism as described herein. In some embodiments, the microorganism is cultured with carbohydrate(s) under defined cultivation conditions. In other embodiments, a disclosed microorganism is cultured under conditions that allow high production rates of hydrogen gas, such as by use of the carbohydrate(s). The disclosure thus includes a method of producing hydrogen gas by cultivating a disclosed microorganism. In further embodiments, hydrogen gas is based upon degradation products of saccharified office paper waste.

In most embodiments, a cultivation condition used in a disclosed method includes the use of an aqueous based culture medium, or aqueous environment. In some embodiments, a cultivation condition includes the presence of inorganic salts. In some cases, the salts are in milligram or microgram amounts, such as by addition of exogenous salts to a culture medium. Non-limiting examples of the salts include those containing iron, selenium, molybdenum, nickel, magnesium, zinc, copper, borate and/or cobalt. In other embodiments, a cultivation condition includes the presence of sulfur-containing compounds. Non-limiting examples include ammonium sulfate, cysteine, glutathione, N-acetyl cysteine and/or dithiothreitol, which may be exogenously added to a culture medium for use in a disclosed method.

In further embodiments, a cultivation condition includes redox-active compounds and/or compounds with antioxidant chemical characteristics. In some cases, the amount of such a compound is defined in the culture medium. Non-limiting examples of such a compound include ascorbic acid, cysteine, N-acetyl cysteine and/or glutathione.

A cultivation condition of the disclosure may also include the presence of a gaseous phase above the culture medium. The gas phase may be optionally continuously flushed, or replenished, with a desired gas. In some embodiments, the desired gas does not contain oxygen. In other embodiments, the desired gas is a noble gas, such as argon as a non-limiting example. In alternative embodiments, the gas is flushed in a discontinuous manner, such as at defined times, during the culturing of the microorganism with the desired gas. In further embodiments, the desired gas is bubbled through the aqueous environment, or culture medium. The bubbling may be continuous or discontinuous, such as at defined time points during the culturing of the microorganism.

In some embodiments, the introduction of gas may be used to remove carbon dioxide generated by the cultivation conditions. Alternatively, carbon dioxide may be chemically bound to an absorbent present under the cultivation conditions. In some cases, the absorbent is an alkali metal liquid matrix. Non-limiting examples include sodium hydroxide (NaOH), and/or a solid matrix, such as soda lime.

A cultivation condition of the disclosure also includes a temperature, and a pH level, suitable for the growth and/or propagation of the microorganism as well as hydrogen gas production. In some embodiments, the temperature is maintained at or below about 45° C. In other embodiments, the pH is maintained at a level from about 4.5 to about 7.5, such as at about 5.0, about 5.5, about 6.0, about 6.5, or about 7.0.

A cultivation condition of the disclosure may also include the continuous supplying of a liquid feedstock, or medium, to the microorganism. In some embodiments, the feedstock contains at least one component selected from monosaccharides, disaccharides, polysaccharides, alcoholic sugars, amino acids, fatty acids, and combinations thereof. Non-limiting examples of monosaccharides and disaccharides include glucose, sucrose, maltose, cellobiose, other saccharides containing glucose units, or any combination of the foregoing. In some embodiments, a feedstock contains arabinose, xylose, galactose, rhamnose, mannitol or any combination of the foregoing.

Therefore, an additional aspect of the disclosure is a culture medium or formulation for use in a method as described herein. The medium or formulation may be a complex or enriched, or alternatively defined or synthetic, growth media which supports hydrogen gas production by a disclosed microorganism. In some embodiments, the medium or formulation allows maximum, as compared to other media or formulations, hydrogen gas production under the conditions used. In other embodiments, the medium or formulation is the defined or synthetic which allows for maximum hydrogen gas production.

In a further aspect, the disclosure includes a method of producing energy. The method may comprise producing hydrogen gas with a disclosed microorganism and supplying the hydrogen gas to a hydrogen gas energy converting device. Non-limiting examples include a fuel cell, gas turbine, internal combustion engine or other suitable conversion device. The converting device may convert the hydrogen gas to either kinetic energy or potential energy. Kinetic energy is based on motion including that of waves, electrons, atoms, molecules, substances, and objects. Non-limiting examples of kinetic energy include electrical energy, radiant energy, thermal energy, motion energy, and sound. Potential energy is stored energy and the energy of position. Non-limiting examples of potential energy include chemical energy, stored mechanical energy, nuclear energy, and gravitational energy.

In a yet further aspect, the disclosure includes a method of identifying, or detecting a disclosed microorganism. In some embodiments, the method comprises identifying or detecting a microorganism as comprising a 16S rDNA sequence containing a sequence with more than 92% homology to SEQ ID No: 1 (Table 5). Non-limiting examples include identifying or detecting a microorganism as comprising a 16S rDNA containing SEQ ID No: 1. In other embodiments, the method comprises identifying or detecting a microorganism as containing a sequence which is amplified by a pair of primers comprising sequences represented by SEQ ID No: 2 and SEQ ID No: 3 (Table 6) from SGT 06-1™ DNA. The method may comprise use of the two sequences as the primers in a polymerase chain reaction (PCR) with DNA from a candidate microorganism followed by comparison of the amplified sequence with that amplified from SGT 06-1™. Non-limiting examples include comparison of the length or base composition of the amplified nucleic acid, or of the sequence of amplified nucleic acid. Optionally, the method may further comprise assaying the candidate microorganism for hydrogen gas production.

The method of identifying or detecting may be of a candidate microorganism isolated from a naturally occurring source or as it is found in nature. Alternatively, the method may be performed with a candidate microorganism derived from a microorganism disclosed herein. In some embodiments, such a derivative, or mutant, microorganism may be one which occurs with passage of a disclosed microorganism in culture. Alternatively, a derivative microorganism may be the result of intentional mutagenesis of a disclosed microorganism.

So in a yet additional aspect, the disclosure includes a method of mutagenizing, or creating, derivative microorganisms from a disclose microorganism. The method may comprise taking a disclosed microorganism and contacting it with a mutagen. Non-limiting examples of mutagens include mutagenic agents, such as chemical compounds, and radiation. The method may further comprise screening the treated microorganism(s) for an rDNA sequence as described herein and/or production of hydrogen gas. In some embodiments, the screening may comprise detection of increased hydrogen gas production. Non-limiting examples of increased production include an increased rate of production over a given period of time and/or increased total gas production over a given period.

Another aspect of the disclosure includes nucleic acid molecules for use in the methods as described herein. In some embodiments, the molecules are isolated from the cellular, or genomic DNA, environment in which they are normally found. One non-limiting molecule is represented by SEQ ID No: 1 (Table 5). In other embodiments, the molecule may be a vector or plasmid, such as one comprising a molecule represented by SEQ ID No: 1. Other molecules of the disclosure are represented by SEQ ID Nos: 2 and 3 (Table 6).

The details of additional embodiments are set forth in the accompanying drawings and the description below. Other features, objects, and advantages of the embodiments will be apparent from the drawings and detailed description, and from the claims.

### BRIEF DESCRIPTION OF THE DRAWINGS

FIG. 1 a shows the total gas production of SGT 06-1™ in comparison to the known gas producing enterobacteria Citrobacter freundii ATCC 13316, Enterobacter cloacae ATCC 15361, and Enterobacter aerogenes ATCC 13048. FIG. 1b shows the calculated hydrogen production rate of SGT 06-1™ in ml H₂/hour×liter over time. For both studies, bacteria were incubated under batch conditions in complex growth medium containing 1% glucose as carbon source.

FIG. 2a shows the growth curve of SGT 06-1™ in comparison to the enterobacterium Citrobacter freundii ATCC 13316 measured as change in optical density at a wavelength of 600 nm over time. FIG. 2b shows the time-dependent hydrogen production of SGT 06-1™ in comparison to C. freundii ATCC 13316 monitored as fuel cell voltage changes. For both studies, bacteria were incubated under batch conditions in complex growth medium containing 1% glucose as carbon source.

FIG. 3a shows the comparative growth curves of the microorganism SGT 06-1™ in complex media and minimum (synthetic) media measured as change in optical density at a wavelength of 600 nm. FIG. 3b shows the time-dependent hydrogen production of SGT 06-1™ incubated in different complex and minimum (synthetic) media monitored as fuel cell voltage changes. For both studies, SGT 06-1™ was grown in the media in the presence of 1% glucose as carbon source and 20 mM ammonium sulfate as nitrogen source under batch conditions.

FIG. 4a shows the comparative hydrogen production of the microorganism SGT 06-1™ in minimum (synthetic) medium in the presence of the carbon feedstock glucose, cellobiose, maltose, and sucrose (data not shown for sucrose) monitored as fuel cell voltage changes. FIG. 4b shows the comparative hydrogen production of SGT 06-1™ in minimum (synthetic) medium in the presence of the monosugars xylose, arabinose, galactose and rhamnose monitored as fuel cell voltage changes. With the exception of cellobiose (0.5%), the concentration of the sugars under investigation was 1%. Glucose is shown for comparison.

FIG. 5 shows the comparative total gas production of the microorganism SGT 06-1™ in minimum (synthetic) medium in the presence of the carbon feedstock glucose, xylose, and the alcoholic sugars mannitol and sorbitol monitored as time-dependent accumulation of gas in inverted Durham tubes. The concentration of the carbon feedstock under investigation was 1.5%.

FIG. 6a shows the comparative growth curves of the microorganism SGT 06-1™ in complex media and minimum (synthetic) media in the presence of glucose (1%) and office paper-derived sugars (obtained after enzymatic saccharification and ultrafiltration) measured as change in optical density at a wavelength of 600 nm. FIG. 6b shows the time-dependent hydrogen production of SGT 06-1™ incubated in complex and minimum (synthetic) media in the presence of glucose (1%) and office paper-derived sugars monitored as fuel cell voltage changes over time. For both studies, SGT 06-1™ was grown in the media in the presence of 1% glucose or office paper-derived sugars (glucose range between 0.2% and 0.4% glucose) as carbon source under batch conditions.

FIG. 7 shows a representative result of a comparative recA PCR experiment after agarose gel electrophoresis and UV transillumination. PCR was performed with DNA isolated from SGT 06-1™ (06-1; Lane 3), Citrobacter freundii (C.f.; Lane 4) ATCC 13316; Enterobacter aerogenes ATCC 13048 (E.a.; Lane 5) and Enterobacter cloacae ATCC 15361 (E.c.; Lane 6) using SEQ ID No: 2 and SEQ ID No: 3 as recA gene-specific primers. Lane 1 (M) contains digested bacteriophage λ DNA as marker DNA. Lane 2 (−) shows a control recA PCR which did not contain bacterial DNA.

FIG. 8 gives an overview of the molecular components and derived carbohydrates of renewable biomass. SGT 06-1™ was tested with the bolded underlined carbohydrates as a carbon source and shown to generate hydrogen gas.

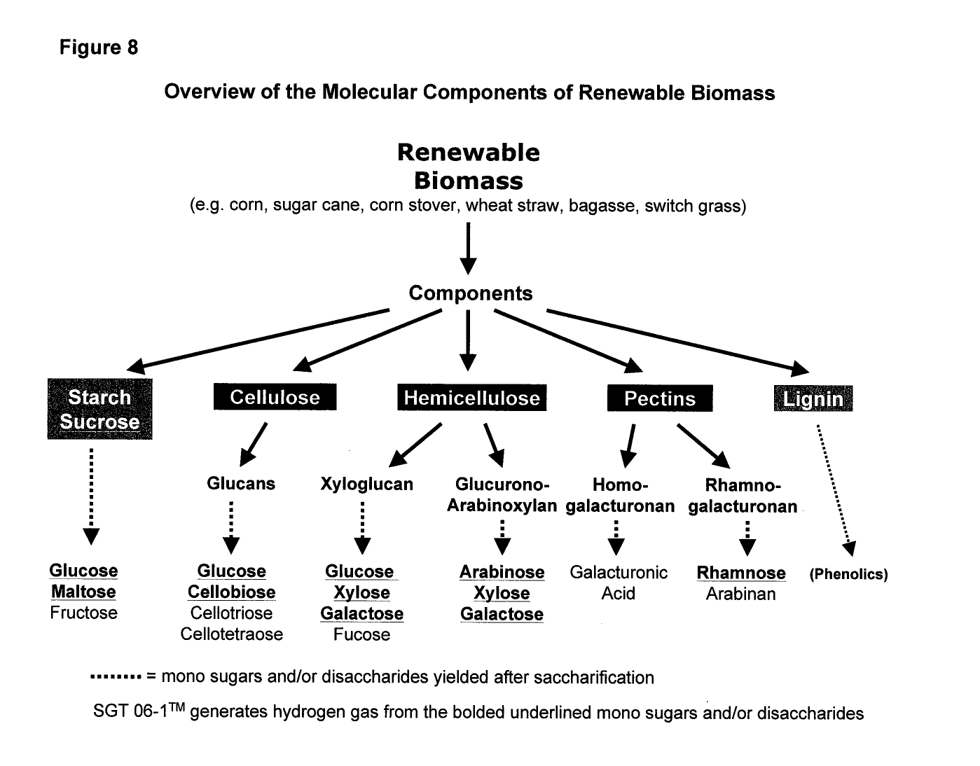

### DETAILED DESCRIPTION OF THE INVENTION

### General

The disclosure is based in part on extensive investigations on an ideal source of hydrogen producing microorganisms and systematically screened environmental samples collected at different locations for high hydrogen producers. The disclosure includes the successful isolation and characterization of a bacterium, termed SGT 06-1™, that has a favorably fast reaction rate and which produces high amounts of hydrogen gas not only from glucose but also from cellobiose, maltose, sucrose, arabinose, xylose, galactose, rhamnose and alcoholic sugars, such as mannitol and sorbitol. Stated differently, the disclosed microorganism is capable of generating hydrogen gas not only from starch and cellulosics-degradation products, such as glucose, maltose and cellobiose, or from hemicellulosics-derived monosugars, such as rhanmose, xylose, galactose and arabinose, but also from alcoholic sugars. Without being bound by theory, and offered to improve the understanding of the disclosed embodiments, the microorganism generates hydrogen gas by fermentation of degradation products of cellulosics materials, such as paper and cotton waste streams, from hemicellulosics degradation products, such as green plant biomass and from alcoholic sugars, such as mannitol, the predominant storage sugar form in brown algae (phaeophytes).

Also without being bound by theory, the isolated and characterized hydrogen gas-generating bacterium termed SGT 06-1™ is believed to belong to the genus Enterobacter. The bacterium, and derivatives thereof as described herein, may be used for long term and large scale generation of hydrogen gas in combination with known or future energy conversion technologies, i.e. fuel cells and/or gas turbines.

The microorganism and methods described herein contributes to the technical field of bio-energy generation from renewable biomass, i.e. sucrose,starch, cellulose- and/or hemicellulose-containing materials. Throughout this document, cellulose-containing materials are herewith referred to as cellulosics, e.g. paper waste, card board, cotton-made fabrics. Hemicellulose-containing materials, e.g. food processing wastes, agriculture and forestry plant biomass, will be termed hemicellulosics. The disclosed microorganism generates hydrogen gas in the presence of structurally diverse carbohydrates, including the monosaccharides glucose, xylose, arabinose, galactose, rhanmose, from the disaccharides cellobiose, maltose and sucrose, from the alcoholic sugar mannitol and also from office paper waste-derived saccharification products. The process described in the disclosed invention is suitable for utilization of sucrose, starch, cellulosics and hemicellulosics-derived carbohydrate streams, as well as waste streams rich in alcoholic sugars, e.g., mannitol, for hydrogen gas production. The industrial scale biohydrogen energy production will utilize the microorganism SGT 06-1™ at sites with traditionally large sucrose, starch, cellulosics- and hemicellulosics waste loads, such as food processing industries, breweries, large office buildings, government offices, educational institutions, shopping malls, hospitals, farms, lumber yards and nurseries. The microorganism may also be utilized for industrial bio-hydrogen production from sources rich in alcoholic sugars, such as brown algae, and at sites with high amounts of alcoholic sugar-containing waste streams containing mannitol and/or sorbitol. The microorganism, cultivation methods and processes of the disclosure can be effectively used for industrial scale production of bio-energy in the form of electricity and/or heat from renewable materials under ultra-low green house gas-emitting conditions. Therefore, the disclosed invention is expected to make significant contributions to air quality improvement, natural resource conservation, land use protection and pollution prevention.

### Microorganisms

As described herein, the disclosure includes a microorganism of the enterobacteriaceae family capable of producing hydrogen gas (H₂) from sucrose different starch, cellulosics- and hemicellulosics-derived carbohydrates, namely glucose, maltose, cellobiose, xylose, rhamnose, galactose and arabinose, in different culture media and under defined cultivation methods. In some embodiments, the hydrogen gas produced by the microorganism SGT 06-1™ is directly used in a fuel cell system for long-term electricity generation under ambient temperatures. Because the microorganism does not produce noxious gases, such as hydrogen sulfide (H₂S) which causes fuel cell membrane poisoning, it is ideal for a fuel cell based energy system.

One non-limiting example of a disclosed microorganism is a hydrogen gas producing microorganism comprising a 16S rDNA sequence represented by SEQ ID No: 1. This microorganism is termed SGT 06-1™, and it has been isolated to be free of other microorganisms found with it in nature. The genetic material of the microorganism may be further isolated and sequenced by methods known to the skilled person to identify additional sequences that are unique, or specific, to the microorganism and/or hydrogen gas production. These additional sequences may also be used to identify or characterize additional hydrogen gas producing microorganisms of the disclosure.

Additional microorganisms of the disclosure include derivatives, or mutants, of SGT 06-1™, such as those which occur spontaneously with its passage or cultivation. In some cases, a derivative microorganism may be considered a progeny microorganism of SGT 06-1™. In other cases, the derivative microorganism is a spontaneous mutant containing genetic changes at one or more locations in the genome of SGT 06-1™. Non-limiting examples of genetic changes includes insertion and/or deletion of sequences, and/or substitution of one or more base residues. In many embodiments, the derivative or mutant microorganism retains the hydrogen gas production phenotype of SGT 06-1™ and/or a 16S rDNA sequence as described herein.

In other embodiments, the additional microorganism may be one which is isolated from its natural environment to be free of one or more microorganisms or components normally found with the microorganism. In some cases, the microorganism may produce hydrogen to the same level as SGT 06-1™.

Whether a derivative, a mutant, or isolated, a microorganism of the disclosure may be identified as comprising a 16S rDNA sequence containing SEQ ID No:1 or a sequence with more than 92% identity to SEQ. ID No: 1. In other embodiments, the microorganism comprises a 16S rDNA sequence containing a sequence with more than 92%, 93%, 94%, 95%, 96%, 97%, 98%, or 99% identity to SEQ ID No: 1. Of course some microorganisms may comprise SEQ ID No: 1. Percent identity between two sequences may be determined by any suitable method as known to the skilled person. In some embodiments, a PSI BLAST search, such as, but not limited to version 2.1.2 (Altschul, S.F., et al., Nucleic Acids Rec. 25:3389-3402, 1997) using default parameters may be used.

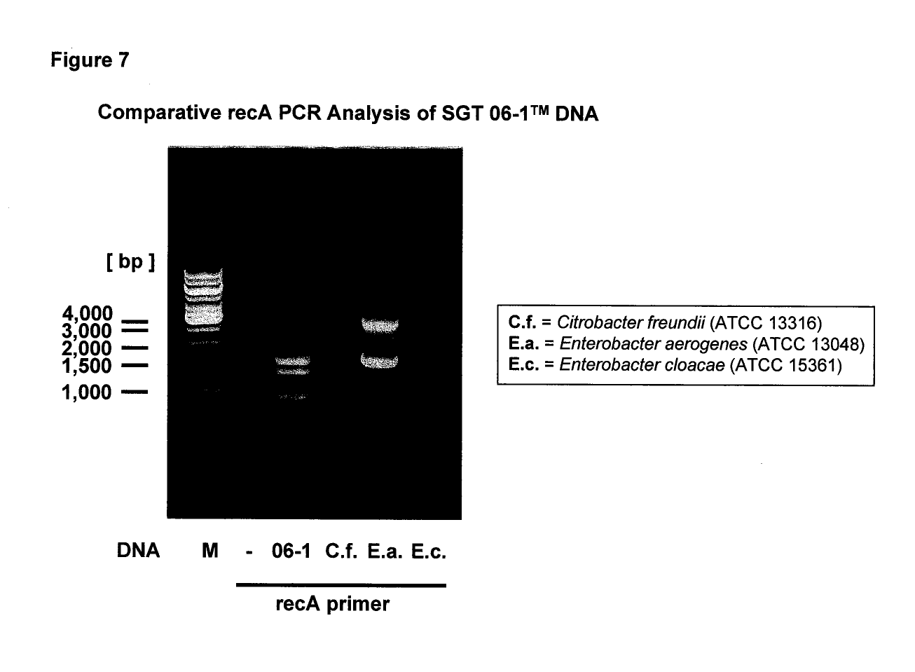

Alternatively, a derivative, a mutant, or isolated microorganism of the disclosure may be identified as comprising a recA sequence amplified from SGT 06-1™ DNA with a pair of primers, one comprising a sequence represented by SEQ ID No:2 and one comprising a sequence represented by SEQ ID No:3. Thus the DNA of a derivative, a mutant, or an isolated microorganism of the disclosure may be amplified by use of the primers, and the amplified nucleic acid molecule may be compared to that amplified from SGT 06-1™ DNA using the same primers. Identity in the pattern of amplified molecules, such as that shown in FIG. 7, identifies the derivative, mutant, or isolated microorganism of the disclosure. Alternatively, identification may be based on the similarities, or identity, in restriction fragment length polymorphism of the amplified molecule, the base composition of the amplified molecule, or the sequence of the amplified molecule to that amplified from SGT 06-1™ DNA.

### Hydrogen Gas Production and Use

In addition to culturing a disclosed microorganism with a suitable medium and conditions to propagate it, the disclosure also includes a method of culturing a microorganism as described herein to produce hydrogen gas. In some embodiments, the microorganism is cultured with a source of carbohydrate(s) as described herein. The method may also comprise cultivation conditions that are suitable or advantageous to hydrogen gas production, such as the use of a culture medium and/or conditions as described herein.

The disclosure thus includes a cell culture comprising a microorganism of the disclosure and a culture medium or formulation as described herein. In some embodiments, the medium or formulation includes the combination of a source of carbohydrate(s), one or more inorganic salts, a sulfur-containing compound, and a redox-active compound and/or antioxidant compound, each of which is as described herein. In further embodiments, the cell culture may be exposed to an absorbent for carbon dioxide as described herein.

The cell culture may be maintained or propagated under conditions that include a combination of a gaseous phase above the medium or formulation, a suitable temperature, suitable agitation of the medium or formulation and an acceptable pH, each as described herein. In some cases, the gaseous phase comprises an inert or noble gas, which is optionally bubbled through a liquid medium or formulation. Non-limiting examples of a suitable temperature include at or below about 45° C. or about 40° C., about 37° C., about 35° C., about 30° C., or about 25° C.

The disclosure further includes a method of producing energy that comprises releasing energy from hydrogen gas produced by a disclosed method. In some embodiments, the method may comprise delivery of hydrogen gas produced by a disclosed microorganism and supplying the hydrogen gas to a hydrogen gas energy converting device. In some cases, the hydrogen gas releases energy during combustion in the presence of oxygen to form water. In other cases, the energy release occurs via electrochemical conversion, such as in a fuel cell with hydrogen gas as a fuel and oxygen as the oxidant.

### Identification of Microorganisms

The disclosure includes a method of identifying, or detecting a disclosed microorganism based on the nucleic acid sequences of the microorganism, optionally in combination with the detection of hydrogen production by the microorganism. Thus in some embodiments, the method comprises identifying or detecting a candidate or test microorganism as comprising 16S rDNA containing a sequence with more than 92% identity to SEQ. ID No: 1, which identifies them as a microorganism of the disclosure. Microorganisms with such levels of sequence identity have been described herein, and they include a microorganism comprising a 16S rDNA containing SEQ ID No: 1.

In other embodiments, the method comprises identifying or detecting a microorganism as containing a 16S rDNA sequence which hybridizes to SEQ ID No: 1 under “stringent conditions.” Hybridization refers to the interaction between two single-stranded nucleic acids to form a double-stranded duplex molecule. The region of double-strandedness may be full-length for both single stranded molecules, full-length for one of the two single stranded molecules, or not full-length for either of the single-stranded nucleic acids. “Stringent conditions” refer to hybridization conditions comprising, or equivalent to, 68° C. in a solution consisting of 5×SSPE, 1% SDS, 5×Denhardt's reagent and 100 μg/ml denatured salmon sperm DNA followed by washing in a solution comprising 0.1×SSPE, and 0.1% SDS at 68° C., or the above conditions with 50% formamide at 42° C. Stringent condition washes can include 0.1×SSC to 0.2×SSC, 1% SDS, 65° C., for about 15-20 min. A non-limiting example of stringent wash conditions is 0.2×SSC wash at 65° C. for about 15 minutes (see, Sambrook et al., Molecular Cloning—A Laboratory Manual (2nd ed.) Vol. 1-3, Cold Spring Harbor Laboratory, Cold Spring Harbor Press, NY, 1989, for a description of the SSC buffer). Other exemplary stringent conditions include 7% SDS, 0.25 M sodium phosphate buffer, pH 7.0-7.2, 0.25 M sodium chloride at 65° C. to 68° C. or such conditions with 50% formamide at 42° C.

In further embodiments, the method comprises amplifying DNA from a test or candidate microorganism with a pair of oligonucleotides, wherein one oligonucleotide comprises a sequence represented by SEQ ID No:2 and the second oligonucleotide comprises a sequence represented by SEQ ID No:3. This pair of oligonucleotides may be used as primers for PCR mediated amplification of an amplicon from a test or candidate microorganism for comparison with an amplicon amplified from SGT 06-1™ using the same primers and PCR conditions. Identity between the amplicons of the test or candidate microorganism compared to SGT 06-1™ allows identification of the test or candidate microorganism as a microorganism of the disclosure. The comparison between the amplicons may be by any means known to the skilled person, including the methods described herein. Optionally, the test or candidate microorganism may already have been identified as producing hydrogen gas.

The test or candidate microorganism may isolated from a naturally occurring source or as found in nature. Alternatively, the method may be performed with a progeny microorganism derived from SGT 06-1™. Alternatively, a derivative microorganism may be the result of intentional mutagenesis of a disclosed microorganism.

### Mutagenesis Methods

The disclosure includes a method of mutagenizing, or creating, derivative microorganisms from a disclose microorganism, such as SGT 06-1™. The method may comprise taking a disclosed microorganism and treating it with a mutagen. Non-limiting examples of a mutagen include UV or ionizing irradiation, a deaminating agent (such as nitrous acid), sodium azide, an intercalating agent (such as ethidium bromide), or phage or transposon mediated mutagenesis. In some embodiments, the method may further comprise the screening of the treated microorganism with a method described herein for the identification of microorganisms by detection of 16S rDNA sequences and/or recA sequences, optionally in combination with detection of hydrogen gas production.

In further embodiments, the mutagenesis method may be used to generate mutated, or altered, microorganisms for identification of microorganisms with increased production of hydrogen gas, relative to SGT 06-1™, as a phenotype. Thus the method may comprise contacting a disclosed microorganism with a mutagen and then screening the treated microorganism for increased hydrogen gas production in comparison to SGT 06-1™. Non-limiting examples of increased production include an increased rate of production over a given period of time and/or increased total gas production over a given period.

### Mutagenesis Methods

The disclosure includes isolated nucleic acid molecules for use in the methods as described herein. The nucleic acid molecule may be DNA in structure, and optionally single or double stranded. In some embodiments, the molecule may include one or more non-conventional bonds, such as an amide bond as found in peptide nucleic acids (PNA). In one embodiment, a nucleic acid molecule comprises, or consists of, a sequence represented by SEQ ID No:1.

In another embodiment, a nucleic acid molecule may be an oligonucleotide comprising, or consisting of, a sequence represented by SEQ ID No:2 or 3. Such a pair of oligonucleotides may be used as primers for a PCR reaction as described herein. In alternative embodiments, the primers may comprise additional sequences at one or both of their 5′ ends that do not interfere with their use in the PCR reaction. In some cases, the additional sequence may be in the form of from about 1 to about 5 additional nucleotides at the 5′ end. In other embodiments, the primers may comprise additional bases at one or both of their 3′ ends that participate in the base pair complementarity with the template molecule in PCR. Thus in some cases, the additional bases are complementary to the corresponding recA sequence.

Having now generally described the invention, the same will be more readily understood through reference to the following examples which are provided by way of illustration, and are not intended to be limiting of the disclosed invention, unless specified.

### EXAMPLES

### Example 1

### General

### Environmental Sampling

The microorganism of the disclosed invention was found in the U.S.A. The sampling site was 42° C., and the natural pH at the site was recorded at 7.2. Samples (5 ml) were aseptically taken from the site and transferred into sterile vials for transportation.

### Growth Medium, Isolation and Cultivation

A small volume (0.5 ml) of the collected sample was inoculated in 25 ml of a sterile basic growth medium containing 6 g Tryptone, 3 g yeast extract, 10 g glucose, 0.3 g MgSO₄, 0.02 g CaCl₂, 67 mM K₂HPO₄ /NaH₂PO₄ buffer, pH 7.0 in liter distilled water. The inoculated sample was incubated at the recorded temperature in a shaker water bath at 120 rpm for 10 to 12 hours. A sample of this culture was streaked on an agar plate prepared with the same basic growth medium and incubated for 24 hours at 37° C. in a humified incubator. Single colonies of the plates were picked, re-inoculated in basic growth medium and restreaked on agar plates. Single colonies of these plates were picked and tested for hydrogen production as described below.

### Example 2

### Measurement of Gas and Hydrogen Production

Picked individual colonies were tested for gas production using inverted Durham test tubes filled with basic growth medium as described above and using the BBL enterotube testing system. During this screening effort a microorganism (SGT 06-1™) was discovered as the bacterium with the highest total gas production within a time period of less than 24 hours.

High hydrogen gas production of the microorganism was measured and achieved using following experimental set-up and incubation conditions. An over night culture of SGT 06-1™ was inoculated into 50 ml of modified basic growth medium as above or alternatively minimum (synthetic) medium and placed into a 250 ml vessel, e.g. Erlenmeyer flask (Pyrex quality). After closing the incubation vessel with a two-way inlet rubber stopper, the content of the vessel was made anaerobic by vacuum-emptying it for 10 minutes with the help of a vacuum pump and by subsequent flushing of the flask with pure argon gas for 10 minutes at a flow rate of 25 ml/min. The incubation vessel with the inoculated bacteria was placed in a shaking water bath or stirred fermentation platform and incubated at 37° C.

Time-dependent generation of hydrogen gas in the vessel was monitored with the help of a linked fuel cell system (Hydro-genius™, HelioCentris, Berlin, Germany). The hydrogen gas-induced increase in current and voltage at the fuel cell was recorded with the help of a fuel cell-connected amperemeter (DT 830B multimeter) and voltmeter (Fluke 10 multimeter). The SGT 06-1™ microorganism was found to be a rapid and high hydrogen producer. The discovered hydrogen producing SGT 06-1™ microorganism was further characterized and its cultivation conditions optimized for maximum and long term hydrogen production under continuous flow conditions.

### Example 3

### Biochemical Analysis and Strain Identification

An isolated colony of the SGT 06-1™ microorganism was inoculated into basic growth medium and grown at 37° C. for 24 hours. Routine examinations and cell counting were performed with a compound light microscope (Olympus, Japan). Gram-staining, which was performed by the Hucker method, revealed the high hydrogen gas producing microorganism as a gram-negative, motile, non-sporulating and non-capsulated short rod which grows under aerobic and anaerobic growth conditions (see ‘General Properties’ in Table 1).

Based upon the further examined biochemical properties of the isolated microorganism using the BBL Enterotube II system (BD Diagnostics, U.S.A.) and individual biochemical tests (see Table 2), the biochemical profile of SGT 06-1™ is almost identical to the reported features of the enterobacterium Citrobacter freundii (see Bergey's Manual of Determinative Bacteriology; 9th edition). The bacterium, however, shows interesting variations to the previously reported Citrobacter strains, including a much faster growth, a clearly different colony morphology and a positive Voges-Proskauer (acetoin) reaction, a negative Methyl-Red reaction and growth and gas production in the presence of lactose as carbon source (see grey shaded areas in Table 2).

TABLE 1
GENERAL PROPERTIES

| | |
|---|---|
| Strain | Growth Ability |

| | |
|---|---|
| Gram | Aerobic |

| | |
|---|---|
| Staining | Anaerobic |

| | |
|---|---|
| Shape | Tryptone-Yeast |

| | |
|---|---|
| Mobility | Water |

| | |
|---|---|
| Spore | Tryptone-Yeast |

| | |
|---|---|
| Capsule | Water |

(+ Glucose)

Minimum
Medium

Minimum
Medium
(+ Glucose)

SGT 06-1

| | |
|---|---|
| negative | yes |

| | |
|---|---|
| short rod | yes |

| | |
|---|---|
| motile | yes* |

| | |
|---|---|
| non-spore | yes* |

| | |
|---|---|
| no | no* |

yes*
*= under aerobic growth conditions; 1% glucose as carbon source

TABLE 2
BIOCHEMICAL PROPERTY

| | SGT 06-1 ™ | C.f.* | E.c.* |
|---|---|---|---|
| Strain | | | |
| Gram stain | − | − | − |
| Spore stain | − | − | − |
| Motility | + | + | + |
| Indole | − | − | − |
| Voges-Proskauer (Acetoin) | + | − | + |
| Methyl Red | − | + | − |
| Citrate | + | + | + |
| Gas (f. Glucose) | + | + | + |
| H₂S | − | −/+ | − |
| Urease (after 24 hrs.) | − | −/+ | −/+ |
| Phenylalanine deaminase | − | − | − |
| Lysine decarboxylase | − | − | − |
| Ornithine decarboxylase | + | −/+ | + |
| Oxidase | − | − | − |
| Catalase | + | + | + |
| Growth & Acid (aerobic)#: | | | |
| D-Glucose | + | + | + |
| D-Adonitol | − | − | −/+ |
| L-Arabinose | + | + | + |
| Cellobiose | + | −/+ | + |
| Dulcitol | − | −/+ | −/+ |
| Lactose | − | −/+ | + |
| Sucrose | + | −/+ | + |
| Maltose | + | + | + |
| D-Xylose | + | + | + |
| D-Mannose | + | + | + |
| D-Sorbitol | + | + | + |
| D-Mannitol | + | + | + |
| L-Rhamnose | + | + | + |
| D-Galactose | + | n/a | n/a |
| Starch | − | n/a | n/a |
| Cellulose | − | n/a | n/a |

*C.f. = Citrobacter freundii; E.c. = Enterobacter cloacae; information based on Bergey's Manual of Determinative Bacteriology' (9th edition)
n/a = data not available in Bergey's Manual of Determinative Bacteriology
#concentration for all carbohydrates under investigation = 0.5%

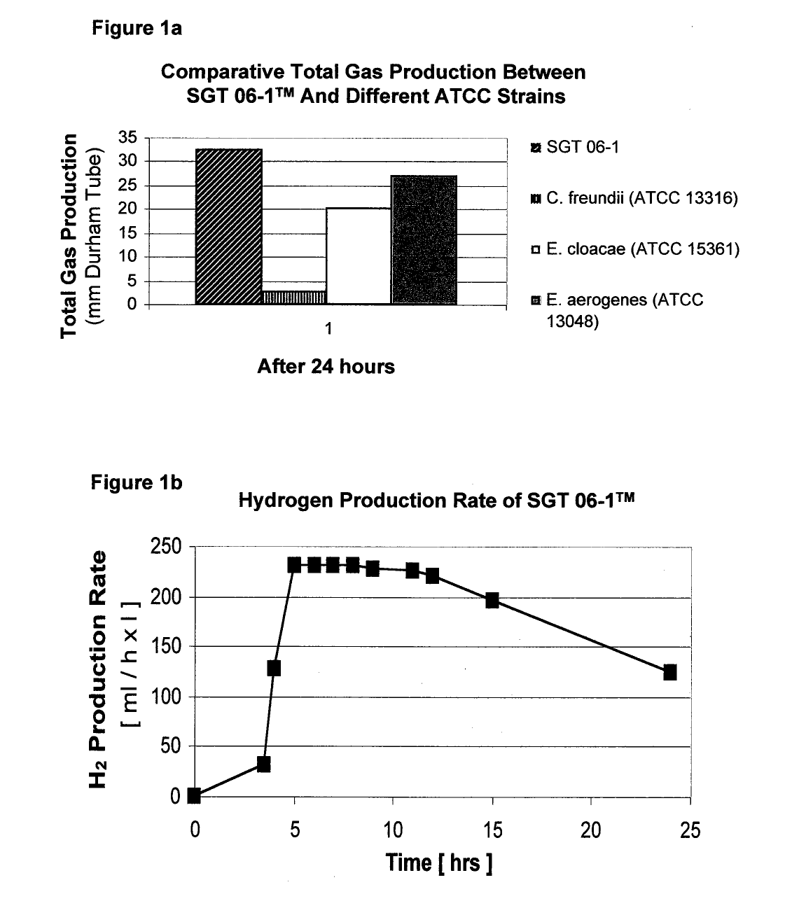

The very high gas production of SGT 06-1™, especially under anaerobic conditions, as indicated by the BBL Enterotube system analysis was further analyzed. To directly compare the total gas production of SGT 06-01™ in nutrient broth (with glucose as feedstock) with other known and commercially available hydrogen producing bacteria, SGT-06-1™, Citrobacter freundii (C.f. ATCC13316), Enterobacter cloacae (E.c. ATCC 15361) and Enterobacter aerogenes (E.a.ATCC 13048) were incubated in parallel in Durham test tubes and monitored for gas accumulation over 24 hours at 37° C. The results of this set of experiments, which are shown as mm gas accumulation in the inverted Durham tube in FIG. 1a confirm the high gas production of SGT 06-1™. The total gas production of SGT-06-1™ is 10 times higher than for C. freundii ATCC 13316, almost twice as high as observed with E. cloacae ATCC 15361 and about 22% higher than for E. aerogenes ATCC 13048. Similar high gas production under anaerobic conditions was observed by incubating the newly isolated microorganism in Durham test tubes with basic growth medium (peptone-salt-glucose) or glucose minimum (synthetic) medium (results not shown). Using a graduated Smith tube the maximum total gas production rate of SGT-06-1™ in basic growth medium (tryptone-yeast-glucose) was measured to be 393 ml gas produced per hour per liter (ml/h×l). This rate is higher than the total gas production rates reported for many hydrogen producing microorganisms under similar batch conditions (for comparison see Table 3) and almost reaches the high gas production rates reported by Taguchi et al. (U.S. Pat. No. 5,350,692) for the anaerobic microorganisms AM21B (650 ml gas/h×l) and AM37F (610 ml gas/h×l) in peptone-yeast glucose (PYG) medium.

The high hydrogen gas production under anaerobic incubation conditions was confirmed by detecting the produced hydrogen gas accumulating in the gas phase with the help of a vessel-connected and calibrated fuel cell system (Hydro-genius™, HeliCentris, Berlin, Germany). Two digital multi-meters were used to measure the hydrogen gas-generated voltage (Fluke 10 multimeter) and current (DT 830B multimeter) of the fuel cell. For this, SGT 06-1™ was inoculated in a 250 ml rubber stoppered flask in 25 ml growth medium (complex or minimum (synthetic) medium). The flask content was made anaerobic by 1) application of a vacuum for 10 min and 2) by subsequent flushing of the gas phase with argon gas for 5 min at a flow rate of 50 ml/min. One of the flask outlets was connected to the fuel cell via flexible gas-tight silicone tubing. Under optimized anaerobic batch culture conditions and in the presence of 1% glucose, SGT 06-1™ shows a very high hydrogen production rate of about 240 ml H₂ /h×l in complex medium (=modified basic growth medium) (FIG. 1b). The hydrogen production rate of the microorganism is comparably high with other known and reported hydrogen generating microorganisms (Table 3). Comparing the hydrogen production rate of SGT 06-1™ with the published rates of other known hydrogen producing microorganisms indicates that the high hydrogen gas productivity of SGT 06-1™ is suitable for industrial scale production of hydrogen gas.

TABLE 3
COMPARISON OF HYDROGEN PRODUCTION RATES

| | Hydrogen Gas Rate | |
|---|---|---|
| Species* | (ml H₂/h × 1) | References |
| Enterobacter sp. SGT 06-1 ™ | 243 | Schmid E. et al. |
| | | (unpublished results) |
| Klebsiella oxytoca | 87.5 | Minnan L. et al., Res. Microbiol. |
| | | 156(1): 76-81 (2005) |
| Citrobacter freundii | 101 | Kumar G. R. et al., Indian J. Exp. |
| | | Biol. 27(9): 824-825 (1989) |
| Enterobacter aerogenes | 246 | Tanisho S. et al., J. Chem. Eng. |
| | | (Japan) 16: 529ff (1983); |
| | | Tanisho S. et al., Int. J. Hydrogen |
| | | Energy 12: 623ff (1987) |
| Enterobacter aerogenes | 120 | Yokoi H. et al., J. Ferment. |
| | | Bioeng. 80: 571ff (1995) |
| Clostridium beijerinckii | 210 | Taguchi F. et al., United States |
| | | Patent #5,350,692 (Sep. 27, |
| | | 1994) |
| Clostridium butyricum | 75 | Ogino H. et al., Biotechnol. Prog. |
| | | 21(6): 1786-1788 (2005) |
| Mixed Anaerobes | 230 | Iyer P. et al., Appl. Microbiol. |
| | | Biotechnol. 66: 166-173 (2004) |
| Mixed bacterial cultures | 74.7 | Van Ginkel S. et al., Environ. Sci. |
| | | Technol. 35(24): 4726-4730 |
| | | (2001) |
| Thermotoga elfii | 125 | Van Niel E. W. J. et al., Hydrogen |
| | | Energy 27: 1391-1398 (2002) |
| Caldicellulosiruptor- | 250 | Van Niel E. W. J. et al., Hydrogen |
| saccharolyticus | | Energy 27: 1391-1398 (2002) |
| Caldicellulosiruptor- | 250 | Kadar Z. et al., Appl. Biochem. |
| saccharolyticus | | Biotechnol. 113-116: 497-508 |
| | | (2004) |
| Thermotoga neapolitana | 460 | Van Ooteghem S. A. et al., Appl. |
| | | Biochem. Biotechnol. 98-100: |
| | | 177-189 (2002) |

*All microorganisms cultivated in batch cultures in the presence of glucose

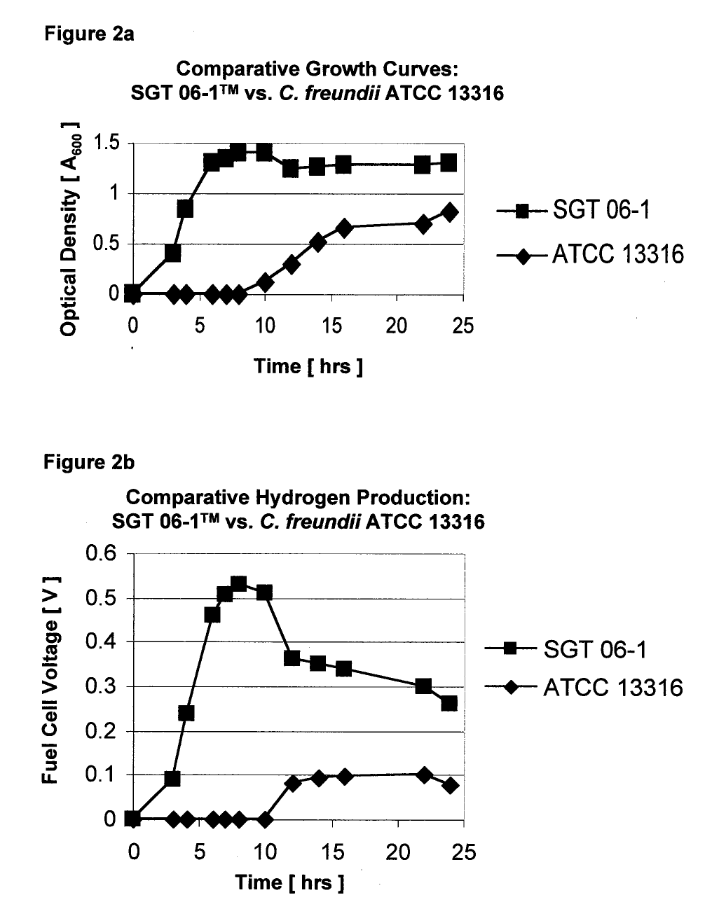

In addition to the favorably high hydrogen production rate, the isolated microorganism SGT 06-1™ shows very rapid cell growth which reaches high cell densities with quick hydrogen gas generation in the presence of glucose when compared with other known hydrogen generating enterobacteria, i.e. Citrobacter freundii ATCC 13316 (see FIG. 2a). SGT 06-1™ reaches maximum hydrogen production in less than 8 hours in tryptone-yeast-glucose medium, while C. freundii ATCC 13316 takes about 15 hours incubation time in the same medium to reach a significantly lower hydrogen production.

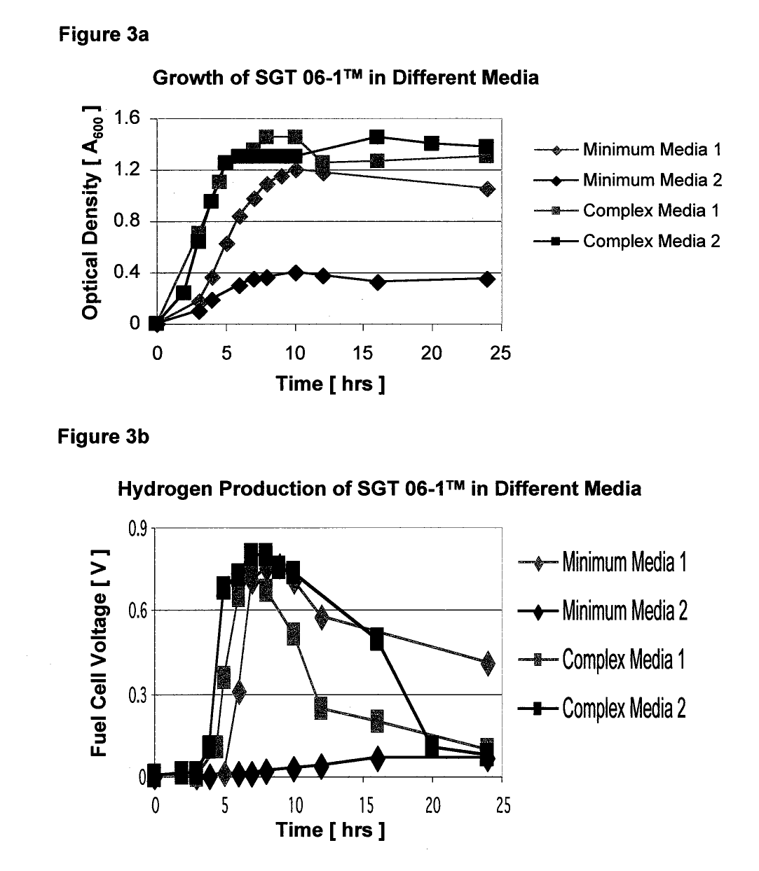

To achieve high hydrogen production rates of the microorganism, different low cost minimum (synthetic) media were tested to support optimum growth and maximum hydrogen production of SGT 06-1™. A successful minimum (synthetic) medium (=minimum medium 1) was derived, which allows growth of SGT 06-1™ to comparatively high cell densities (FIG. 3a) and leads to comparably high hydrogen production rates (FIG. 3b) as previously observed in complex media. Following specific minimum (synthetic) media formulation has been demonstrated as suitable to support growth and high production by SGT 06-1™.

### Aqueous Minimum (Synthetic) Medium (CMM):

| | | |
|---|---|---|
| H₂O | 1.0 | L |
| Na₂HPO₄ | 6 | g |
| KH₂PO₄ | 3 | g |
| NH₄SO₄ | 2 | g |
| NaCl | 0.5 | g |
| MgSO₄•7H₂O | 0.1 | g |
| CaCl₂•2H₂O | 0.01 | g |
| Trace element solution | 1.0 | ml |
| Feedstock, e.g. glucose | 15 | g |

The following trace element solution was devised to supplement the minimum (synthetic) medium for high hydrogen production of SGT 06-1™.

### Trace Element Solution

| | | |
|---|---|---|
| H₂O | 1.0 | L |
| FeCl₃•6H₂O | 1.5 | g |
| Na₂SeO₃•5H₂O | 15 | mg |
| Na₂MoO₄•H₂O | 36 | mg |
| NiCl₂•6H₂O | 24 | mg |
| ZnCl₂ | 70 | mg |
| MnSO₄•6H₂O | 0.1 | g |
| CuCl₂•2H₂O | 2 | mg |

SGT 06-1™ grows and produces acids not only in the presence of the carbohydrate glucose, but also when inoculated in complex and/or minimum (synthetic) media in the presence of other carbohydrates as feedstock, such as sucrose, cellobiose, lactose, arabinose, rhamnose, galactose, xylose, sorbitol, mannitol, mannose, maltose and trehalose (data not shown). SGT 06-1™ was not able to grow in the presence of the sugars D-adonitol and dulcitol.

### Example 4

### High Hydrogen Production in Different Media

Growth of the microorganism SGT 06-1™ and its capacity to generate high amounts of hydrogen gas with glucose as carbon feedstock was tested with different complex and minimum growth media (FIG. 3a and 3b). For this, SGT 06-1™ was inoculated in 50 ml each of either basic or modified tryptone-yeast-glucose liquid media (=complex media 1 and 2) or basic or modified minimum (synthetic) glucose liquid media (=minimum media 1 and 2) in 250 ml rubber stoppered flasks in an argon atmosphere. The concentration of glucose in the flasks was 1%. Generation of hydrogen gas—which peaked at around 7-8 hours cultivation time—was measured every hour as hydrogen gas-generated voltage changes on a connected fuel cell system. With the exception of minimum growth medium 2, SGT 06-1™ grows equally well and reaches comparatively high cell densities in complex media 1 and 2 as well as in minimum (synthetic) medium 1 (FIG. 3a). Maximum cell densities are reached after less than 10 hours incubation time at a temperature of 37° C. With the exception of minimum (synthetic) medium 2, SGT 06-1™ produces equally high amounts of hydrogen gas in both complex media and in minimum (synthetic) medium 1 as indicated by comparatively high voltage generation via the incubation vessel-connected fuel cell (FIG. 3b). The observation where SGT 06-1™ is capable of generating high amounts of hydrogen gas not only during incubation in more expensive complex media but also when incubated in minimum (synthetic) growth media conditions, is of key importance for a cost-effective large scale hydrogen energy production using the microorganism.

### Example 5

### Hydrogen Production from Sucrose, Cellobiose and Maltose

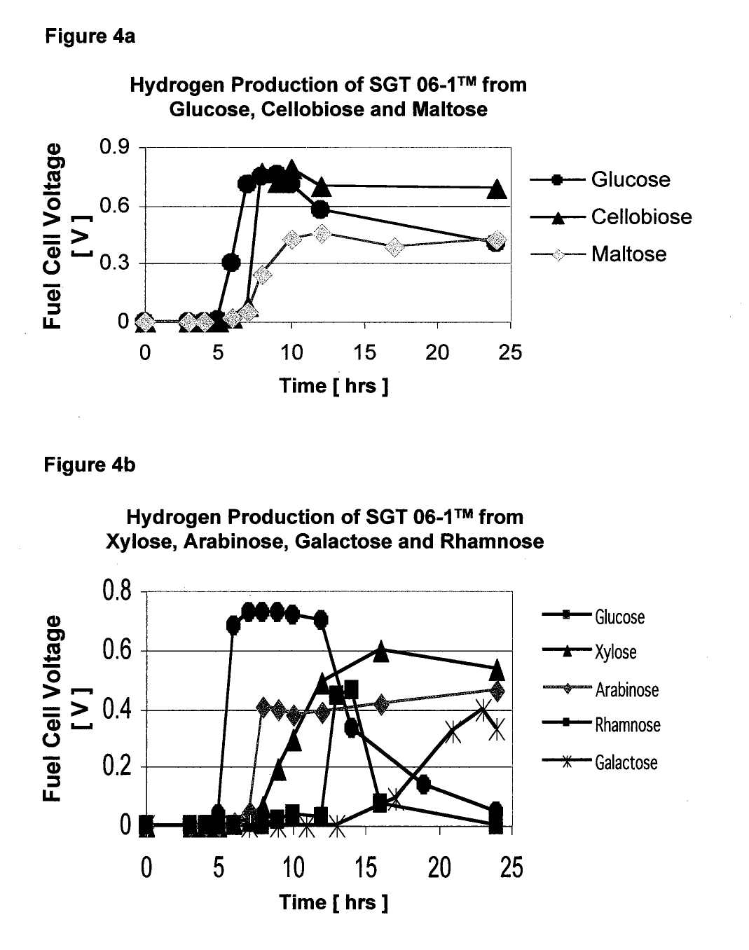

Next the hydrogen production capacity of SGT 06-1™ was tested in the presence of carbohydrates other than glucose. There was interest in whether SGT 06-1∩ grows and is capable of generating comparatively high amounts of hydrogen gas in the presence of the important plant disaccharide sucrose as well as from the cellulose- and starch-derived disaccharides cellobiose and maltose. As shown in FIG. 4a, SGT 06-1™ shows strong growth and high hydrogen production not only in the presence of glucose, but also when incubated in the presence of cellobiose, maltose, and sucrose (data not shown for sucrose). This set of experiments proved that the microorganism SGT 06-1™ not only generates high amounts of hydrogen gas from glucose—as indicated by high voltage readings of the connected fuel cell system—but also when grown in the presence of the disaccharides sucrose, cellobiose and maltose. It is of interest that the hydrogen production of SGT 06-1™ is much more prolonged when grown in the presence of cellobiose and maltose than in glucose.

In the presence of cellobiose and maltose as feedstock, high fuel cell readings were consistently observed even after more than 24 hours incubation under batch conditions. This finding, where the isolated SGT 06-1™ is able to generate high amounts of hydrogen gas not only from the monosaccharide glucose as feedstock, but also from the common disaccharides sucrose, cellobiose and maltose, is of high commercial value. It allows simplified and cost saving future industrial scale hydrogen production from traditionally high sucrose-containing wastes, e.g. bagasse and food industry wastes, maltose-containing waste streams, e.g. brewery wastes, and from the common cellulosics materials derived degradation product cellobiose. The latter observation will allow future industrial scale microbial hydrogen production from cellobiose as feedstock rather than glucose under conditions which will only require a single enzyme (with exoglucanase activity) for cost effective preparation of cellobiose feedstock from cellulosics materials.

### Example 6

### Hydrogen Production from Hemicellulosics Sugars

A farther set of studies was conducted for the capability of SGT 06-1™ to generate high quantities of hydrogen gas in the presence of monosugars found in typical hydrolysates of hemicellulosic materials, such as xylose, arabinose, galactose and rhamnose. As shown in FIG. 4b, SGT 06-1™ generates high amounts of hydrogen gas not only when incubated in the presence of 1% glucose, but also in the presence of 1% xylose, 1% rhamnose, 1% arabinose and 1% galactose. It is of interest that hydrogen production by SGT 06-1™ shows a distinctive prolonged lag phase in the presence of the hemi-cellulosics-associated sugars when compared with glucose. With xylose and galactose as carbon source maximum hydrogen generation is observed after 15 hours which continues to remain at a relatively high level even after 24 hours.

### Example 7

### Gas Production from Alcoholic Sugars

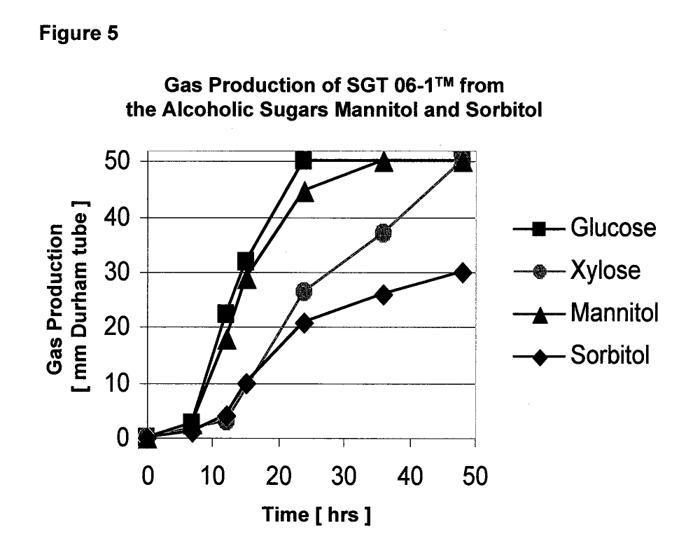

Yet another important set of studies conducted was the capability of SGT 06-1™ to generate high quantities of hydrogen gas in the presence of a unique set of sugars, called alcoholic sugars, such as mannitol and sorbitol. As shown in FIG. 5, SGT 06-1™ generates high amounts of gas in the presence of the alcoholic sugars mannitol and sorbitol within 24 hours incubation time. Glucose and xylose are shown for comparison in this Figure. The observation where SGT 06-1™ is capable of generating high amounts of hydrogen gas not only from cellulosics-and hemicellulosics-derived sugars, such as glucose and xylose, but also from the alcoholic sugars mannitol and sorbitol, makes this microorganism ideal for industrial scale generation of hydrogen energy from sources and waste streams rich in these alcoholic sugars, such as brown algae and nutritional industry.

### Example 8

### Hydrogen Production from Office Paper-derived Sugars

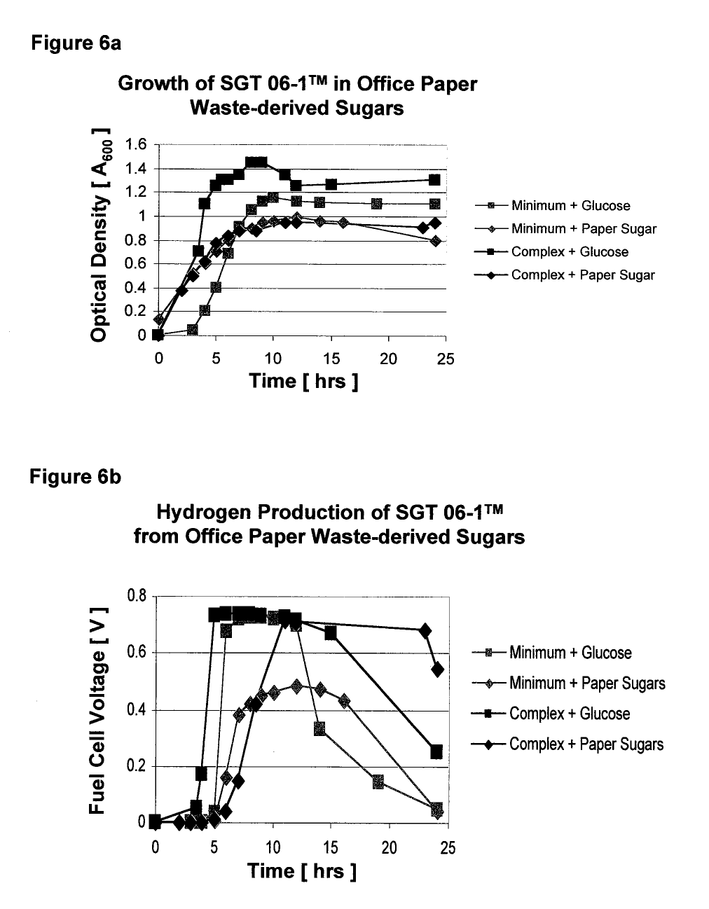

Yet another important set of studies conducted was the capability of SGT 06-1™ to generate high quantities of hydrogen gas in the presence of sugars derived from office paper waste after saccharification. For this, collected office paper waste was pretreated and enzymatically converted into reduced sugars using a purified and commercially available fungal cellulase following steps and procedures known in the arts of cellulosics saccharification. As shown in FIG. 6a, SGT 06-1™ grows well in the presence of office paper-derived sugars (=paper sugar) and reaches almost as high optical densities as when grown in the presence of glucose. Comparable optical densities can be reached with SGT 06-1™ either growing in modified tryptone-yeast medium (=complex medium) or in modified minimum (synthetic) medium (=minimum medium).

More significantly, SGT 06-1™ under identical incubation conditions in complex medium—generates equivalent amounts of hydrogen gas in the presence of paper sugars as when incubated in the presence of glucose (FIG. 6b). Microorganism SGT 06-1™ is also capable of generating high amounts of hydrogen gas from paper sugars when incubated in minimum (synthetic) medium (FIG. 6b). It is of interest that hydrogen production of SGT 06-1™ in paper sugars shows a slightly longer lag phase and is significantly longer when compared with glucose. With office paper-derived sugars as carbon source, SGT 06-1™ still generates high amounts of hydrogen gas after more than 20 hours incubation time under the chosen batch conditions. The observation where SGT 06-1™ is capable of generating high amounts of hydrogen gas from office paper-derived sugars makes this microorganism ideal for industrial scale generation of hydrogen energy from paper- and other primarily cellulosics-comprised waste streams.

### Example 9

### PCR and 16SrRNA Gene Sequence Analysis

Due to the encountered difficulty to precisely assign the newly isolated microorganism SGT 06-1™ to the genus Citrobacter freundii within the enterobacteria family using conventional biochemical testing systems, molecular biological methods were used to identify the isolate by 16S-rRNA gene sequence analysis. For direct comparison and for serving as an internal control of the following procedure, PCR with DNA isolated from a commercially available Citrobacter freundii strain (ATCC 13316) and Enterobacter aerogenes (ATCC13048) strain was used as an internal standard and control of the applied method.

PCR-dependent 16S rRNA gene sequence analysis was carried out as follows. Isolates were grown in basic growth medium A for 20-24 hours at 37° C. and genomic DNA was isolated from pellets of collected bacterial cells using the Qiagen spin column method. A 701 bp fragment of the 16S rRNA gene of the isolated genomic DNA of SGT 06-1™ was amplified by PCR using a designed universal 16S rRNA primer pair (SGT-UNI04fw3 and SGT-UNI04rv2 (see Table 4 below). SGT-UNI04fw3 and SGT-UNI04rv2 recognize highly conserved nucleotide sequences of the GenBank-deposited 16S rDNA sequence (nucleotide 140-160; nucleotide 824-841) of Citrobacter freundii ATCC 29935 (gi: 174064), and span a hypervariable region of the C. freundii 16S rRNA gene.

TABLE 4
USED PCR PRIMER FOR 16S rDNA ANALYSIS OF SGT 06-1 ™

SGT-UNI04-fw3....................5′-TGGAGGGGGATAACTACTGG-3′ (SEQ ID No:4)
SGT-UNI04-rv2....................5′-GGCACAACCTCCAAGTCG-3′ (SEQ ID No:5)

Twenty picomoles of forward primer (SGT-UNI04-fw3) and reverse primer (SGT-UNI04-rv2) were used in the PCR reaction. The PCR reaction mixture further contained 0.5 units Taq polymerase (Invitrogen), 500 ng of genomic DNA, 0.1 mmol/l of each nucleotide (dNTPs) and 1.5 mM MgCl₂, in a total volume of 20 μl. A 701 bp fragment of the 16S rRNA gene was amplified after 35 cycles in an automated thermal cycler (MyCycler, BioRad) using following temperature profile: [4 min at 95° C.; (30 s at 95° C., 30 s at 56° C., 2 min at 72° C.)₃₅ₓ; 5 min at 72° C.].

After separation by low melting agarose gel electrophoresis, the 16S-rRNA PCR product was excised and purified with use of the Qiagen gel purification kit. The base sequence of the purified 701 bp 16S rRNA gene segment was determined by using the Tag Dye Deoxy Terminator Cycle Sequencing method (Seqxcel Inc., San Diego, Calif.) and compared with the nucleotide sequences deposited with the NCBI (National Center for Biological Information) database (all GenBank+EMBL+DDBJ+PDB sequences). A comparative analysis of the retrieved 654 base sequence (see Table 5) of SGT 06-1™ was done with the GenBank database using NCBI BLAST (blastn & MegaBlast). It revealed that SGT 06-1™ is related to gram-negative bacteria showing highest sequence similarity to members of the enterobacteriaceae family. Top scoring sequence similarities were reported for the 16S rRNA gene sequences of the following databank-deposited microorganisms [rankings based on lowest Expect (E) values and highest maximum score]:

1. Enterobacter sp. CBMB30
(gi: 56392829; Accession #: AY683044.1)

| | |
|---|---|
| Maximum Score: 800 | E Value = 0.0 |

2. Uncultured gamma proteobacterium clone MTG-9
(gi: 83415738; Accession #: DQ307725.1)

| | |
|---|---|
| Maximum Score: 778 | E Value = 0.0 |

3. Uncultured gamma proteobacterium clone MTG-6
(gi: 83415730; Accession #: DQ307717.1)

| | |
|---|---|
| Maximum Score: 778 | E Value = 0 |

4. Enterobacter sp. FMB-1
(gi: 113869742; Accession #: DQ855282.1)

| | |
|---|---|
| Maximum Score: 767 | E Value = 0.0 |

Parallel base sequence analysis of a 16S rRNA gene fragment amplified from isolated DNA of a Citrobacter freundii strain (ATCC 13316) and of a Enterobacter aerogenes strain (ATCC13048) was performed using PCR and BLAST analysis as controls to test the validity of the chosen 16S rRNA PCR parameters and procedure. Using the BLAST blastn tool, the the Citrobacter freundii and Enterobacter aerogenes base sequence retrieved after automated dideoxynucleotide sequencing (Seqxcel Inc., San Diego, Calif.) were compared with the sequences deposited with the NCBI (National Center for Biological Information) database (all GenBank+EMBL+DDBJ+PDB sequences). This search confirmed the validity of the gene sequencing procedure used and produced top scoring sequence hits with the 16S rDNA of 18 different Citrobacter freundii strains all with an E Value of 0.0 (with PCR-amplified ATCC13316C. freundii 16S rDNA as submitted sequence) and of 5 different Enterobacter aerogenes strains all with an E Value of 0.0 and with high maximum scores of larger than 1275 (with PCR-amplified ATCC13048 Enterobacter aerogenes 16S rDNA).

Summarized, genomic DNA was isolated from SGT 06-1™, and the base sequence has been successfully analyzed with an obtained 701 bp 16S rDNA fragment. The microorganism SGT 06-1™ is believed to belong to the enterobacteriaceae family based on this analysis. Because the level of 16S rDNA gene identity with their closest taxonomically named relatives was less than 93%, and due to reported variations in several biochemical characteristics with Enterobacter species (see grey-shaded areas in Table 2), most prominently E. aerogenes and E. cloacae, the isolated microorganism is believed to be an Enterobacter and perhaps represents a new species based on the presented unique biochemical and genetic features. Via the 16S rDNA gene analysis, which closely related the isolated microorganism to the two enterobacterial species Enterobacter sp. CBMB30 (Accession No. AY683044.1) and Enterobacter sp. FMB-1 (Accession No. DQ855282.1) the microorganism is named Enterobacter sp. SGT 06-1™ for further reference and preliminary classification. The isolated microorganism Enterobacter sp. SGT 06-1™ was deposited with the American Type Tissue Collection (ATCC) on Jun. 12, 2007 and was given the ATCC No: PTA-8465.

TABLE 5
BASE SEQUENCE OF 16S rRNA GENE FRAGMENT OF
SGT 06-1 ™(SEQ ID No:1)

GATAATCGCA TACGTCGCAG ACCAAGAGGG GGACCTTCGG
GCCTCTTGCC
ATCAGATGTG CCCAGATGGG ATTAGCTAGT AGGCGGGGTA
ATGGCCCACC
TAGGCGACGA TCCCTAGCTG GTCTGAGAGG ATGACCAGCC
ACACTGGAAC
TGAGACACGG TCCAGACTCC TACGGGAGGC AGCAGTGGGG
AATATTGATC
AATGGGCGCA AGCCTGATGC AGCCATGCCG CGTGTGTGAA
CAAGGCCTTC
GGATTGTAAA TCGCTTTCTC CGGGTAGGAA GGCCTGCCGG
TTAATAACCG
TGCCGATTGA CTTTACCCGC AGAAGAAGCA CCGGCTAACT
CCGTGCCGCA
CCCGCTTCCT CCCCGAGGGT GCAAGCGTTA ATCGGAATTA
CCTTCTTCAA
AGCGCACGCA TGGCTGCTGT CAAGTTGGAT GTGAAATCCC
CGGGCTCAAC
CTGCTAACTG CATTCGAAAC TGGGAGGCTG GAGTCTCGTA
GAGGGAGGTG
GAATTCCTCG TGTACCGGTG AAATGCCTAC ACCTCTGGAG
GACTATCCCC
GGCCAACGCG GTCTCCTGGA CTAAGACTGA CTCTCAGGTG
CAAAACCGTG
GGGACCCCAC TTGATTATAT ACCCTGGTAG TCCACTCCGC
TAACGATGTC
AACTTGATGC TCCCTTCAAA
(670 bp)

### Example 10

### PCR and recA Gene Sequence Analysis

Further genotypic characterization of the newly isolated SGT 06-1™ was undertaken to confirm the genomic difference of SGT 06-1™ to other known enterobacterial species. For this, a primer pair (SEQ ID No. 2 and SEQ ID No. 3) was designed based on low variability sequence regions of the Erwinia carotovora recA gene deposited in the NCBI nucleotide data base (on the worldwide web at ncbi.nlm.nih.gov). The recA gene, which codes for recA, a multifunctional protein involved in homologous recombination, DNA repair and the bacterial SOS response, has been suggested to serve as a molecular marker suitable for genotyping and identification of bacteria (Ludwig W. et el., ASM News 65: 752-757 (1999)). Because the recA gene has been successfully used for genetic typing of a variety of bacteria, including Erwinia acinetobacters, and mycobacterial species (Waleron M. et al., Microbiology 148: 583-595 (2002)), the disclosed recA primer pair was used for PCR-based genetic analysis of SGT 06-1™ DNA.

TABLE 6
USED PCR PRIMER FOR RecA Gene ANALYSIS OF SGT 06-1 ™

SEQ ID No.2..........................5′-GGTAAAGGGTCTATCATGCG-3′
SEQ ID No.3..........................5′-CCTTCACCATACATAATTTGGA-3′

RecA-PCR was performed with DNA isolated from the microorganism SGT 06-1™ as well as with DNA from three commercially available enterobacter species, i.e. Citrobacter freundii strain (ATCC 13316), Enterobacter aerogenes (ATCC 13048) and Enterobacter cloacae (ATCC 15361).

PCR-based DNA amplification of an expected 735 bp fragment of the recA gene was carried out in 20 μl reaction volumes using the “Ready-To-Go” PCR bead technology (GE Healthcare), including 20 pmol of each recA primer and 50-100 ng of isolated bacterial DNA. DNA fragments of the recA gene were amplified after 35 cycles in an automated thermal cycler (MyCycler, BioRad) using following temperature profile: [4 min at 95° C.; (1 min at 94° C., 1 min at 47° C., 2 min at 72° C)₃₅ₓ; 5 min at 72° C.].

The recA PCR amplicons were separated by agarose gel electrophoresis, visualized with the help of a UV transilluminator and documented with a Polaroid camera system. RecA-PCR of which a typical result is shown in FIG. 7 indicates strong genetic differences between the bacterial species examined as indicated by significantly different DNA fragments received after recA PCR. The recA PCR pattern of the novel isolated bacterium SGT 06-1™ which displays three typical PCR amplification products, does not match with any of the recA PCR products retrieved with Citrobacter freundii (ATCC 13316), Enterobacter aerogenes (ATCC 13048) and Enterobacter cloacae (ATCC 15361) DNA. The results of the recA PCR analysis experiments prove the usefulness of this method and the used recA primer pair (=SEQ ID No:2 and SEQ ID No:3) for sensitive PCR-based detection and identification of the isolated new enterobacterial species.

All references cited herein, including patents, patent applications, and publications, are hereby incorporated by reference in their entireties, whether previously specifically incorporated or not.

Having now fully provided the instant disclosure, it will be appreciated by those skilled in the art that the same can be performed within a wide range of equivalent parameters, concentrations, and conditions without departing from the spirit and scope of the disclosure and without undue experimentation.

The disclosure has been described in connection with specific embodiments thereof, it will be understood that it is capable of further modifications. This application is intended to cover any variations, uses, or adaptations of the disclosure following, in general, the disclosed principles and including such departures from the disclosure as come within known or customary practice within the art to which the disclosure pertains and as may be applied to the essential features hereinbefore set forth.

### Bibliography

### U.S. Patents:

| | | | |
|---|---|---|---|
| 2429589 | October, 1947 | Wiley | 435/167. |
| 3383309 | May, 1968 | Chandler | 48/197. |
| 3711392 | January, 1973 | Metzger | 435/167. |
| 3764475 | October, 1973 | Mandels et al. | 435/209. |
| 4480035 | October, 1984 | Roychowdhury | 435/168. |
| 5350692 | September, 1994 | Taguchi et al. | 435/252.7 |
| 6887692 | December, 2002 | Paterek JR | 438/168. |
| 6860996 | March 2005 | Noike et al. | 210/603. |
| 6942998 | September, 2005 | Ooteghem | 435/168. |

What is claimed is:

1. An isolated microorganism deposited at ATCC under accession no. PTA-8465.

2. An isolated, hydrogen producing microorganism comprising a 16S rDNA sequence fragment represented by SEQ ID No: 1.

3. An isolated, hydrogen producing microorganism comprising a 16S rDNA sequence containing a sequence with more than 92% homology to SEQ. ID No: 1.

4. A derivative or mutant of the microorganism of claim 1 comprising a 16S rDNA sequence containing a sequence with more than 92% homology to SEQ ID No: 1.

5. The microorganism of claim 4 wherein said 16S rDNA sequence comprises SEQ ID No: 1.

6. A pair of oligonucleotides, wherein one oligonucleotide comprises a sequence represented by SEQ ID No: 2 and the second oligonucleotide comprises a sequence represented by SEQ ID No: 3 used for identification of a hydrogen producing microorganism according to claim 1.

7. A method of producing molecular hydrogen (H₂), said method comprising culturing the microorganism according to claim 1 under conditions allowing hydrogen production.

8. The method of claim 7 wherein said conditions comprise an aqueous environment containing microgram amounts of added inorganic salts, such as iron, selenium, molybdenum, nickel and zinc, and/or milligram amounts of added sulfur-containing compounds, such as ammonium sulfate, N-acetyl cysteine, cysteine, and/or dithiothreitol.

9. The method of claim 7 wherein said conditions comprise an aqueous environment containing defined amounts of redox-active compounds and/or compounds with antioxidant chemical characteristics, such as ascorbic acid, N-acetyl cysteine, cysteine and/or glutathione.

10. The method of claim 7 wherein said conditions comprise a gas phase above an aqueous environment that is continuously flushed with defined amounts of a gas, such as the noble gas argon.

11. The method of claim 10 wherein the gas phase above the aqueous environment is flushed at defined time points with defined amounts of a gas, preferentially the noble gas argon.

12. The method of claim 7 wherein said conditions comprise an aqueous environment that is continuously bubbled with defined amounts of a gas, such as the noble gas argon.

13. The method of claim 7 wherein said conditions comprise an aqueous environment that is flushed at defined time points with defined amounts of a gas, such as the noble gas argon.

14. The method of claim 7 wherein said conditions comprise an environment maintained at a temperature below 45° C.

15. The method of claim 7 wherein said conditions comprise an environment that is maintained at a constant pH of between 4.5 and 7.5.

16. The method of claim 7 wherein said conditions comprise a continuously supplied liquid feedstock derived from the group consisting of monosaccharides, disaccharides, polysaccharides, alcoholic sugars, amino acids, glycerol, fatty acids, and combinations thereof.

17. The method of claim 16 wherein the mono- and disaccharides are glucose, sucrose, maltose, cellobiose and/or other saccharides containing glucose units or combination thereof.

18. The method of claim 16 wherein the feedstock contains arabinose, xylose, galactose, and/or rhanmnose, sorbitol and/or mannitol or combinations thereof.

19. The method of claim 7 wherein the conditions comprise generation of carbon dioxide which is chemically bound with the help of an alkali metal liquid matrix, such as sodium hydroxide (NaOH), and/or a solid matrix, such as soda lime.

20. A method of producing energy, said method comprising producing hydrogen according to claim 7 and supplying said hydrogen to a hydrogen gas energy converting device, such as a fuel cell, gas turbine, or internal combustion engine.

21. A method of mutagenizing the microorganism of claim 1, said method comprising the treatment of said microorganism with a mutagen, optionally further comprising screening said treated microorganism for increased hydrogen production rates and/or output.

22. The method of claim 21 wherein the mutagen is UV or ionizing irradiation, a deaminating agent, an alkylating agent, sodium azide, an intercalating agent, or phage or transposon mediated mutagenesis.

23. The method of claim 22 wherein the deaminating agent is nitrous acid, the alkylating agent is methyl-N-nitrosoguanidine (MNNG) and the intercalating agent is ethidium bromide.
